(19) United States
(12) Patent Application Publication
(10) Pub. No.: US 2017/0135832 A1
(43) Pub. Date: May 18, 2017

(54) DEVICES, SYSTEMS, AND METHODS TO PRECONDITION, ARTERIALIZE, AND/OR OCCLUDE A MAMMALIAN LUMINAL ORGAN

(71) Applicant: CVDevices, LLC, San Diego, CA (US)

(72) Inventor: Ghassan S. Kassab, La Jolla, CA (US)

(73) Assignee: CVDevices, LLC, San Diego, CA (US)

(21) Appl. No.: 15/417,952

(22) Filed: Jan. 27, 2017

Related U.S. Application Data
(63) Continuation-in-part of application No. 15/023,394, filed on Mar. 20, 2016.
(60) Provisional application No. 62/333,060, filed on May 6, 2016.
(60) Provisional application No. 62/287,568, filed on Jan. 27, 2016.
(60) Provisional application No. 61/882,837, filed on Sep. 26, 2013.

(51) Int. Cl. A61F 2/93; A61F 2/95; A61B 17/12

(52) U.S. Cl. CPC A61F 2/93; A61B 17/12036; A61B 17/1204; A61B 17/12109; A61B 17/12172; A61F 2/95; A61B 2017/1205; A61F 2230/0069; A61F 2210/0061

(57) ABSTRACT

Devices, systems, and methods to precondition, arterialize, and/or occlude a mammalian luminal organ. In an exemplary embodiment of a device, the device comprises a frame comprising a plurality of struts and configured for expansion, and a covering surrounding at least an external perimeter of the frame, the covering comprising an impermeable material, wherein the device is configured for expansion without requiring the introduction of a gas and/or a liquid therein.

### RELATED APPLICATIONS

The present patent application a) is related to, and claims the priority benefit of, U.S. Provisional Patent Application Ser. No. 62/333,060, filed May 6, 2016, b) is related to, and claims the priority benefit of, U.S. Provisional Patent Application Ser. No. 62/287,568, filed Jan. 27, 2016, and c) is related to, claims the priority benefit of, and is a U.S. continuation-in-part patent application of, U.S. Nonprovisional patent application Ser. No. 15/023,394, filed Mar. 20, 2016, which is related to, claims the priority benefit of, and is a U.S. national stage patent application of, International Patent Application Serial No. PCT/US2014/057703, filed Sep. 26, 2014, which is related to, and claims the priority benefit of, U.S. Provisional Patent Application Ser. No. 61/882,837, filed Sep. 26, 2013. The contents of each of the foregoing applications are hereby incorporated by reference in their entirety into the present disclosure.

### BACKGROUND

Thousands, if not millions, of patients in the U.S. and across the globe suffer from cardiac, peripheral, and other circulatory conditions to such an extent that further cardiac or other procedures are no longer possible with current medical technology. As such, said patients are at the risk of potential death, loss of peripheral limb function or limbs altogether, or other catastrophic or potentially catastrophic conditions, due to diseases and other conditions within their circulatory systems.

In view of the same, devices and systems useful to precondition, arterialize, and/or occlude a mammalian luminal organ, to address at least the conditions identified above, would be well appreciated in the medical arts.

### BRIEF SUMMARY

In an exemplary embodiment of a device for preconditioning, arterializing, and/or occluding a mammalian luminal organ of the present disclosure, the device comprises a frame comprising a plurality of struts, wherein at least one strut of the plurality of struts forms a local general perimeter or boundary of the device, and an expandable occluder coupled to the frame and comprising a membrane or other expandable material that can generally expand and/or unfold as device shifts from a first configuration to a second configuration. In another embodiment, when the device is deployed within a luminal organ, the device can remain in place within the luminal organ for as long as desired. In yet another embodiment, the first configuration is a generally compressed configuration, and wherein the second configuration is a generally expanded or deployed configuration. In an additional embodiment, the first configuration allows the device to be delivered intravascularly within a patient. In yet an additional embodiment, the expandable occluder is located at or near a distal end of the device.

In an exemplary embodiment of a device for preconditioning, arterializing, and/or occluding a mammalian luminal organ of the present disclosure, the expandable occluder is located at a relative center/middle of the device. In an additional embodiment, when the device is deployed within a luminal organ, the expandable occluder hinders blood flow through the luminal organ at the location of the expandable occluder. In yet an additional embodiment, the expandable occluder comprises a material selected from the group consisting of a biological material and a biologically-compatible material, but not limited to, polytetrafluoroethylene (PTFE), visceral pleura, lung ligament tissue, and other suitable bodily (mammalian) tissues. In another embodiment, the expandable occluder is connected to the frame using one or more sutures. In yet another embodiment, the plurality of struts includes at least one perimeter strut and/or at least one lateral strut used, for example, to minimize blockage of side branches.

In an exemplary embodiment of a device for preconditioning, arterializing, and/or occluding a mammalian luminal organ of the present disclosure, the plurality of struts includes at least two perimeter struts and at least one lateral strut, the at least one lateral strut connected to two of the at least two perimeter struts. In another embodiment, the plurality of struts includes at least three perimeter struts, and wherein the expandable occluder is coupled to the frame at a most distal of the at least three perimeter struts.

In an exemplary embodiment of a device for preconditioning, arterializing, and/or occluding a mammalian luminal organ of the present disclosure, at least part of the device tapers from one end to another end (such as from a first end to an opposite second end). In another embodiment, a first gap exists between two of the plurality of struts, wherein a second gap exists between two of the plurality of struts, and wherein the first gap is larger than the second gap.

In an exemplary embodiment of a device for preconditioning, arterializing, and/or occluding a mammalian luminal organ of the present disclosure, the plurality of struts comprises a first perimeter strut, a second perimeter strut, and a third perimeter strut. In another embodiment, a first gap exists between the first perimeter strut and the second perimeter strut, wherein a second gap exists between the second perimeter strut and the third perimeter strut, and wherein the first gap is larger than the second gap. In yet another embodiment, the device has a proximal end and a distal end, and wherein the expandable occluder is located at or near the distal end. In an additional embodiment, the device is configured so that the proximal end of the device can fit within a first vein and further configured so that the distal end of the device can fit within a branch vein of the first vein. In yet an additional embodiment, the device is configured to fit within an opening of an atrial appendage. In another embodiment, when the device is expanded from a generally compressed configuration to a generally expanded or deployed configuration within the opening of the atrial appendage, the expandable occluder occludes the opening of the atrial appendage. In yet another embodiment, the device further comprises a second expandable occluder coupled to the frame. In an additional embodiment, the device, along with a delivery device, comprises an exemplary system of the present disclosure.

In an exemplary embodiment of a method of using an exemplary device of the present disclosure, the method comprises the steps of inserting an exemplary device of the present disclosure within a non-arterial luminal organ, the exemplary device comprising a frame and an expandable occluder coupled thereto, expanding the exemplary device from a first, collapsed configuration to a second, expanded configuration to anchor the exemplary device within the luminal organ, wherein the exemplary device, when in the second, expanded configuration, is operable to block bodily fluid therethrough at the location of the expandable occluder. In another embodiment, the exemplary device, when expanded and anchored in a vein, facilitates preconditioning of the vein prior to arterialization. In yet another embodiment, the exemplary device, when expanded and anchored in a vein, facilitates localized arterialization at or near the expandable occluder. In an additional embodiment, when the exemplary device is expanded and anchored in a vein at a bifurcation having a first side branch and a second side branch, blood can flow through the first side branch but not the second side branch.

In an exemplary embodiment of a method of using an exemplary device of the present disclosure, the exemplary device, when expanded and anchored in the luminal organ, causes a localized stenosis at or near the expandable occluder. In another embodiment, the step of expanding is performed to cause an occlusion within the non-arterial luminal organ due to expansion of the expandable occluder. In an additional embodiment, the exemplary device, when expanded and anchored in the luminal organ, causes a thrombosis at or near the expandable occluder. In yet an additional embodiment, the exemplary device, when expanded and anchored in the luminal organ, causes an increase in fluid pressure within the luminal organ at or near the expandable occluder. In another embodiment, the exemplary device, when expanded and anchored in the luminal organ, facilitates thickening of walls (considered as functional arterialization) of the luminal organ at or near (such as distal to) the expandable occluder.

In an exemplary embodiment of a method of using an exemplary device of the present disclosure, the exemplary device, when expanded and anchored in an opening of an atrial appendage, prevents blood flow in and/or out of the atrial appendage, such as for atrial fibrillation patients that are prone to thrombus generation in the appendage. In an additional embodiment, the step of inserting is performed to position the device within a vein, and wherein the device, when in the second, expanded configuration, blocks blood flow through the vein at the expandable occluder. In yet an additional embodiment, the device is configured to fully block a flow of bodily fluid in the non-arterial luminal organ at the expandable occluder. In another embodiment, the method further comprises the step of performing a medical procedure after the exemplary device is anchored within the luminal organ. In yet another embodiment, the medical procedure comprises a coronary bypass graft procedure or an anastomosis procedure. In various embodiments, the medical procedure is performed to treat a peripheral circulatory system condition. In at least one embodiment, the plurality of struts includes at least two perimeter struts and at least one lateral strut, the at least one lateral strut connected to two of the at least two perimeter struts, and wherein the step of inserting is performed to insert the device within a non-arterial luminal organ at a bifurcation so that the bifurcation is located at the at least one lateral strut.

In an exemplary embodiment of a device for preconditioning, arterializing, and/or occluding a mammalian luminal organ of the present disclosure, the device comprises a frame comprising a plurality of struts and configured for expansion; and a covering surrounding at least an external perimeter of the frame, the covering comprising an impermeable material; wherein the device is configured for expansion without requiring the introduction of a gas and/or a liquid therein. In at least one embodiment, the device is configured to at least partially occlude a mammalian luminal organ at a location of the device when the device is positioned within the mammalian luminal organ and when the frame is expanded from a compressed configuration to an expanded configuration. In at least one embodiment, the impermeable material comprises rubber.

In an exemplary embodiment of a device for preconditioning, arterializing, and/or occluding a mammalian luminal organ of the present disclosure, the impermeable material comprises a biologically-compatible material. In at least one embodiment, expansion of the frame from a compressed configuration to an expanded configuration causes a diameter or distance of the device to increase. In at least one embodiment, no fluid is present within the device. In at least one embodiment, the device is configured to completely occlude a mammalian luminal organ at a location of the device when the device is positioned within the mammalian luminal organ and when the frame is expanded from a compressed configuration to an expanded configuration.

In an exemplary embodiment of a device for preconditioning, arterializing, and/or occluding a mammalian luminal organ of the present disclosure, the device defines a passageway therethrough, the passageway extending from a first end to a second end of the device, the passageway configured to allow blood to flow therethrough when the device is positioned within a mammalian luminal organ and expanded to at least partially occlude the mammalian luminal organ at the device. In at least one embodiment, a boundary of the passageway is defined by the covering.

In an exemplary embodiment of a device for preconditioning, arterializing, and/or occluding a mammalian luminal organ of the present disclosure, the device forms part of an overall system, the overall system further comprising a delivery cannula configured to attach to and detach from the device. In at least one embodiment, the overall system further comprises a detachment member coupled to the delivery cannula, the detachment member configured so that the delivery cannula can attach to and detach from the device. In at least one embodiment, the delivery cannula comprises a distal portion and a proximal portion, wherein the passageway is configured to receive at least part of the distal portion therethrough, and wherein the distal portion is configured to detach from the proximal portion after the device is positioned within the mammalian luminal organ.

In an exemplary embodiment of a device for preconditioning, arterializing, and/or occluding a mammalian luminal organ of the present disclosure, the device further comprises a retrieval feature coupled thereto or formed therein, the retrieval feature configured for engagement by a retrieval device, whereby the retrieval device can engage the retrieval feature of the device after the device has been positioned within a mammalian luminal organ so to retrieve the device from the mammalian luminal organ. In at least one embodiment, the device comprises a main portion and a proximal portion, whereby the main portion would define a largest expanded cross-sectional area, and whereby the proximal portion generally tapers inward from the main portion toward a proximal end of the device. In at least one embodiment, the device further comprises a distal portion, the distal portion generally tapering inward from the main portion toward a distal end of the device.

In an exemplary embodiment of a device for preconditioning, arterializing, and/or occluding a mammalian luminal organ of the present disclosure, the device comprises a frame comprising a plurality of struts and configured for expansion; and a covering surrounding at least an external perimeter of the frame, the covering comprising an impermeable rubber material; wherein the device is configured for expansion without requiring the introduction of a gas and/or a liquid therein; and wherein the device is configured to at least partially occlude a mammalian luminal organ at a location of the device when the device is positioned within the mammalian luminal organ and when the frame is expanded from a compressed configuration to an expanded configuration.

In at least one embodiment, the device defines a passageway therethrough, the passageway extending from a first end to a second end of the device, the passageway configured to allow blood to flow therethrough when the device is positioned within a mammalian luminal organ and expanded to at least partially occlude the mammalian luminal organ at the device, and wherein a boundary of the passageway is defined by the covering.

In an exemplary embodiment of a method for preconditioning, arterializing, and/or occluding a mammalian luminal organ of the present disclosure, the method comprises the steps of introducing at least part of a device into a mammalian luminal organ, the device comprising a frame comprising a plurality of struts and configured for expansion, and a covering surrounding at least an external perimeter of the frame, the covering comprising an impermeable material, wherein the device is configured for expansion without requiring the introduction of a gas and/or a liquid therein, and expanding the frame to at least partially occlude the mammalian luminal organ. In at least one embodiment, the step of introducing is performed by introducing the at least part of the device into the mammalian luminal organ using a cannula coupled to the device. In at least one embodiment, the method further comprises the step of retrieving the at least part of the device from the mammalian luminal organ using a retrieval device.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a side view of a device in a compressed configuration, according to an exemplary embodiment of the present disclosure;

FIG. 2A shows a side view of a device in an expanded configuration, according to an exemplary embodiment of the present disclosure;

FIG. 2B shows a perspective view of a device in an expanded configuration, according to an exemplary embodiment of the present disclosure;

FIG. 3 shows a device positioned within a luminal organ, according to an exemplary embodiment of the present disclosure;

FIG. 4 shows an anastomosis, according to an exemplary embodiment of the present disclosure;

FIG. 5A shows a device used to occlude an atrial appendage, according to an exemplary embodiment of the present disclosure;

FIG. 5B shows a component block diagram of a system, according to an exemplary embodiment of the present disclosure;

FIG. 6 shows a perspective view of a device in an expanded configuration and having a self-sealing element therein, according to an exemplary embodiment of the present disclosure;

FIG. 7A shows a side view of a device in a compressed configuration, according to an exemplary embodiment of the present disclosure;

FIG. 7B shows a side view of a device in an expanded configuration, according to an exemplary embodiment of the present disclosure;

FIG. 8A shows a distal portion of a delivery cannula having a balloon and a wire positioned therein, according to an exemplary embodiment of the present disclosure;

FIG. 8B shows a distal portion of a delivery system, according to an exemplary embodiment of the present disclosure;

FIG. 9A shows at least part of a device in a compressed or collapsed configuration, according to an exemplary embodiment of the present disclosure;

FIG. 9B shows at least part of a device in an expanded configuration, according to an exemplary embodiment of the present disclosure;

FIG. 10 shows at least part of an expanded device having a passageway defined therethrough, according to an exemplary embodiment of the present disclosure;

FIG. 11A shows at least part of a device coupled to a delivery cannula, according to an exemplary embodiment of the present disclosure;

FIG. 11B shows at least part of a device coupled to a delivery shaft, according to an exemplary embodiment of the present disclosure;

FIG. 11C shows at least part of a device coupled to a delivery cannula, in an expanded configuration within a luminal organ, according to an exemplary embodiment of the present disclosure;

FIG. 12 shows at least part of an expanded device positioned within a luminal organ and having a distal portion of a delivery cannula therein, according to an exemplary embodiment of the present disclosure;

FIG. 13 shows a block component diagram of a device having a retrieval feature, according to an exemplary embodiment of the present disclosure;

FIG. 14 shows at least part of a device having a main portion, a tapered distal portion, and a tapered proximal portion, according to an exemplary embodiment of the present disclosure;

FIG. 15 shows at least part of a device having a main portion and a tapered proximal portion, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features, are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

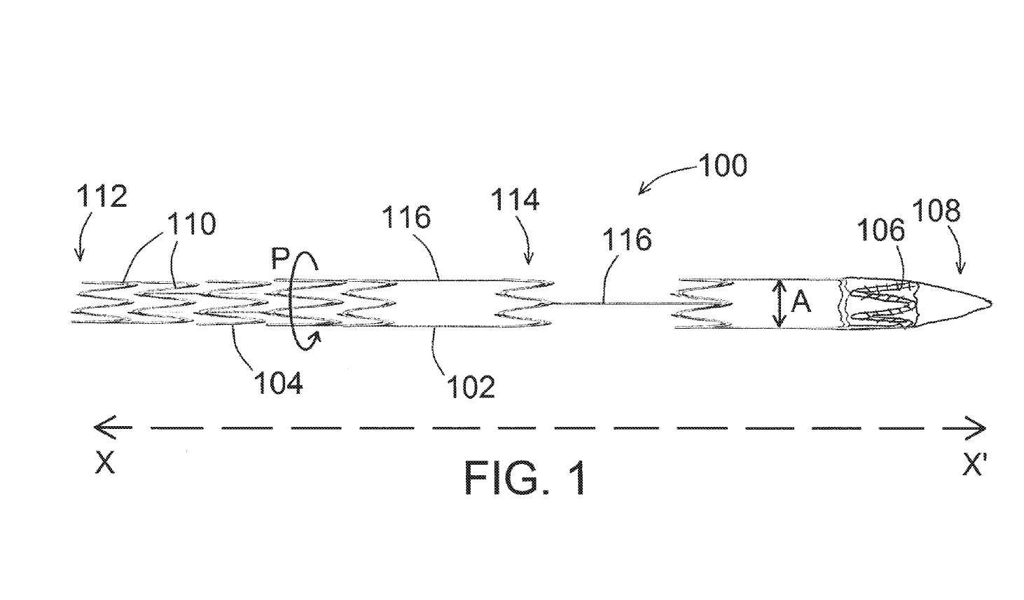

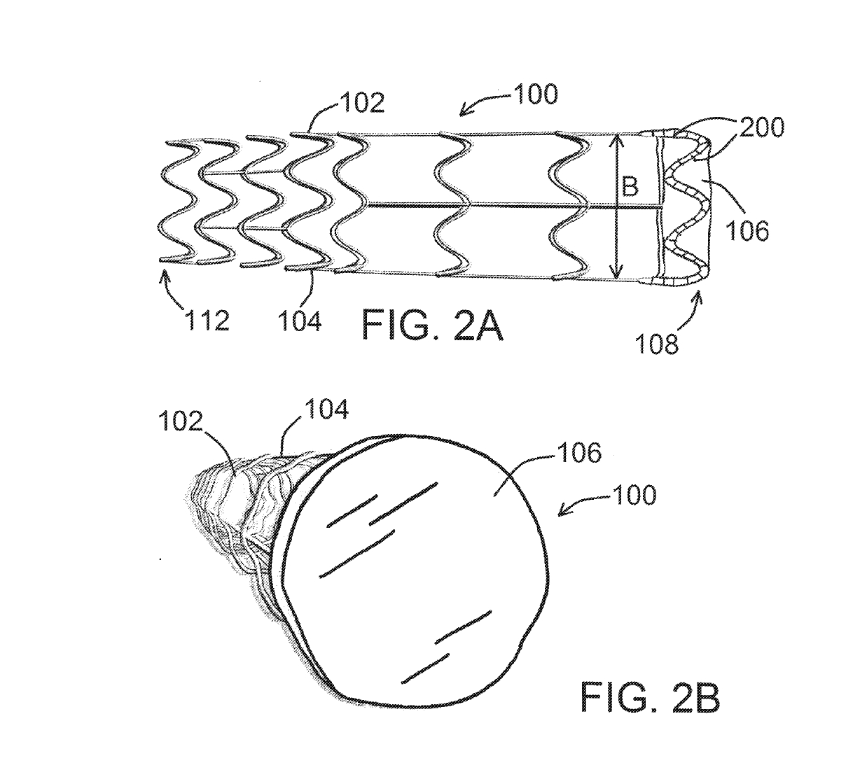

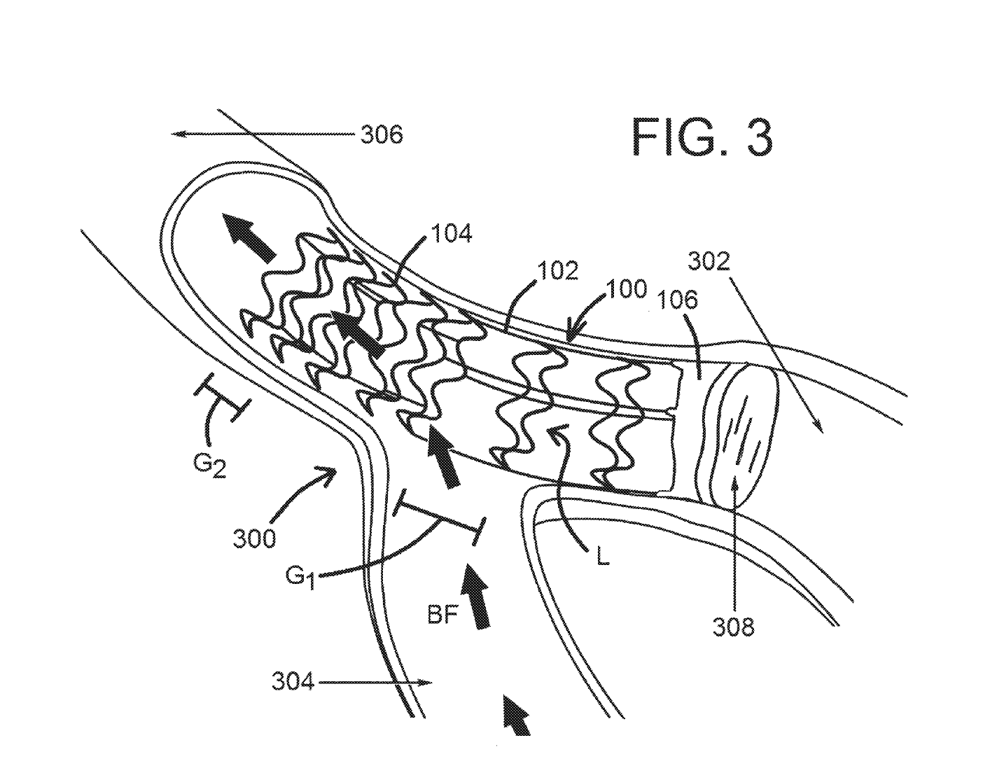

An exemplary device for preconditioning and/or occluding a luminal organ of the present disclosure is shown in FIG. 1. As shown in FIG. 1, device 100 comprises a frame 102 having a number of struts 104, whereby at least one of the various struts 104 forms a local general perimeter or boundary of device 100 (shown as P in the figure) so that device 100, when ultimately deployed within a luminal organ 300 (such as a blood vessel, for example, as shown in FIG. 3), device 100 can remain in place within said luminal organ 300 for as long as desired. Device 100 is shown in a generally compressed configuration in FIG. 1, and is shown in a generally expanded or deployed configuration in FIGS. 2A and 2B. The generally compressed configuration, as referenced in further detail herein, allows device 100 to be delivered intravascularly within a patient and allows the user delivering device 100 to do so without requiring a traditional or open surgical procedure.

Various embodiments of device 100, as shown in FIGS. 1, 2A, and 2B, for example, further comprise an expandable occluder 106 located along device 100. As shown in in FIGS. 1, 2A, and 2B, for example, expandable occluder 106 is located at or near distal end 108 of device 100, but in other embodiments, expandable occluder 106 could be located at a different location of device 100, such as at a relative center/middle of device 100. Expandable occluder 106, in various embodiments, may comprise a membrane or other expandable material that can generally expand and/or unfold as device 100 shifts from the first configuration (relatively or generally compressed) to the second configuration (relatively or generally expanded or deployed) so that upon delivery and expansion into a blood vessel (an exemplary luminal organ 300), blood flowing through device 100 is hindered due to expandable occluder 106. For example, expandable occluder 106 may comprise one or more biological or biologically-compatible materials including, but not limited to, mammalian membrane tissue (such as pulmonary or other ligament tissue, pulmonary or other visceral tissue, etc.), polytetrafluoroethylene (PTFE), and/or other materials known in the art configured to collapse and expand or stretch. Expandable occluder 106, as shown in FIG. 2A for example, may be connected to frame 102 of device 100 using one or more sutures 200.

As shown in FIG. 1, frame 102 of device 100 may comprise several portions/features to facilitate delivery and placement within a luminal organ 300 and/or to direct or facilitate fluid flow therethrough. For example, and as shown in FIG. 1, an exemplary frame 102 may comprise a plurality of perimeter struts 110, whereby said perimeter struts 110 generally or fully form a local perimeter of frame 102, as indicated by P in FIG. 1. Perimeter struts 110 may be positioned along device 100 at locations where it is desired to have frame 102 of device 100 contact a luminal organ 300 wall upon deployment of device 100 to anchor device 100 therein. For example, a plurality of perimeter struts 110 may be positioned at or near a proximal end 112 of device 100, as shown in FIG. 1, to anchor device 100 within luminal organ 300. Perimeter struts 110 may be positioned at various other locations along device 100, such as, for example, at a relative middle 114 of device 100, or elsewhere between proximal end 112 and distal end 108 of device 100. As shown in FIG. 1, for example, perimeter struts 110 may have a wavy (back and forth) pattern, and may extend around part, all, or substantially all of an overall perimeter or boundary (P) of device 100.

An exemplary frame 102 may comprise a number of other types of struts 104 as well. For example, and as shown in FIG. 1, one or more lateral struts 116 may be used, which extend along part of a longitudinal axis (extending from X to X′ as shown in FIG. 1) of device 100, and used to anchor device 100 within luminal organ 300 and/or to allow various portions of device 100 to connect to one another or generally form a unitary device 100. Lateral struts 116, as shown in FIGS. 1 and 2A, for example, may be positioned at different locations along a perimeter of frame 102. Perimeter struts 110 and lateral struts 116, as referenced and shown herein, are each exemplary struts 104 of the present disclosure, but are described and shown in the figures using separate reference numbers so that the various struts configurations can be separately described. In at least one embodiment, for example, at least one perimeter strut 110 and/or at least one lateral strut 116 are used, for example, to minimize blockage of side branches of vessels.

FIGS. 2A and 2B show an embodiment of an exemplary device 100 of the present disclosure in a generally expanded or deployed configuration. As shown therein, expandable occluder 106 is configured to span an entire cross-sectional area (CSA) of device 100 at the location of expandable occluder 106. Expansion/deployment of device 100, as shown between FIG. 1 and FIG. 2A, for example, causes a local diameter to enlarge, such as a first diameter (labeled as “A” in FIG. 1) enlarging to a second diameter (labeled as “B” in FIG. 2A). Furthermore, and in at least one exemplary device 100 embodiment of the present disclosure, device 100 may generally taper on one direction (such as tapering down from distal end 108 to proximal end 112 as shown in FIG. 2A), or in an opposite direction (not shown). The spanned CSA of expandable occluder 106, as shown in FIGS. 2A and 2B, correspond to second diameter B, for example.

An exemplary device 100 of the present disclosure positioned within a patient's vein (an exemplary luminal organ 300 of the present disclosure) is shown in FIG. 3. As shown therein, device 100 is positioned at a venous bifurcation, such as where the left anterior descending vein 302 and the right anterior descending vein 304 merge into the great cardiac vein 306, for example, so that blood flow (identified as BF in FIG. 3) moves in the direction of the bold arrows shown in the figure.

With such a device 100 placement, and as generally referenced herein, various devices 100 of the present disclosure are configured to provide for a localized stenosis, with the overall size/width of the stenosis related to the size/width of the expandable occluder 106 in an expanded configuration. Said devices 100 can be used within a mammalian body to locally block fluid flow, such as locally blocking blood flow through a vein 300, causing a local stenosis and also locally increasing the pressure within the vein at that location to precondition and/or arterialize that portion of the vein for use with an additional procedure as generally referenced herein. As shown in FIG. 3, for example, the stenosis 308, which may also be referred to as a thrombus 308, is quite local as blood can continue to flow through the other venous side branch through struts 104 of device 100 and/or depending on where device 100 is positioned within the body/vasculature. As shown in FIG. 3, various device 100 embodiments of the present disclosure may be configured so that at least one relatively larger gap (identified as G1 in the figure) exists and that at least one relatively smaller gap (identified as G2 in the figure) exists between perimeter struts 110 (and/or other struts 104). As shown therein, at least one relatively larger gap G1 exists, when device 100 is positioned within a luminal organ 300 at a bifurcation, at the bifurcation so that blood or other fluid flow is less hindered by device 100, which would reduce or preclude the likelihood of clotting, as blood or another fluid can travel through that portion of device 100 (such as at gap G1) and into and through a relative lumen L of device 100 prior to flowing into luminal organ 300. Smaller gaps G2 may exist where additional device 100 structural integrity is desired, such as where device 100 contacts luminal organ 300.

Said devices 100 can be delivered and deployed by an interventionalist, such as within a coronary vein of a patient, to precondition (or pre-arterialize) the vein, which can be followed by a coronary venous bypass graft, for example. Such a procedure may be a sole option for patients who would otherwise not have any other options remaining, such as stenting fully diseased arteries or performing a coronary artery bypass graft (CABG) procedure.

As generally referenced herein, various devices 100 of the present disclosure are intended to occlude venous blood vessels and potentially other non-arterial luminal organs within the body. Said devices 100 of the present disclosure can be used to precondition venous vessels for potential grafting as a traditional artery, or potential re-routing of blood flow therethrough. For example, a device 100 of the present disclosure can be positioned within a vein for a desired amount of time (such as two weeks, or a longer or shorter time as desired) to precondition and/or arterialize said vein due to increased local pressure (such as an increase to 40-50 mm Hg, for example), resulting in general venous wall thickening (an example of functional arterialization) at that location, and anastomosis can be performed to the newly arterialized vein, such as shown in FIG. 4. As shown in FIG. 4, a graft 400 can be connected to the arterialized vein (an exemplary luminal organ 300) at an anastomosis location 402, allowing blood to flow into vein (as identified by BF and the bold arrows in FIG. 4), noting that the arterialized vein can now withstand the higher pressures of arterial blood flowing therethrough, and said higher pressure arterial (oxygenated) blood can be routed to a portion of the body as needed/desired, such as to treat an ischemic portion of the body. Said arterialization can be performed within a patient's peripheral venous system, and/or can be used to arterialize a vessel not in a patient's peripheral venous system but ultimately transplanted, via graft, to the patient's peripheral venous system to treat a peripheral limb condition, for example, which may potentially effectively salvage a limb that would otherwise ultimately cease to function and/or require amputation, such as a patient's foot.

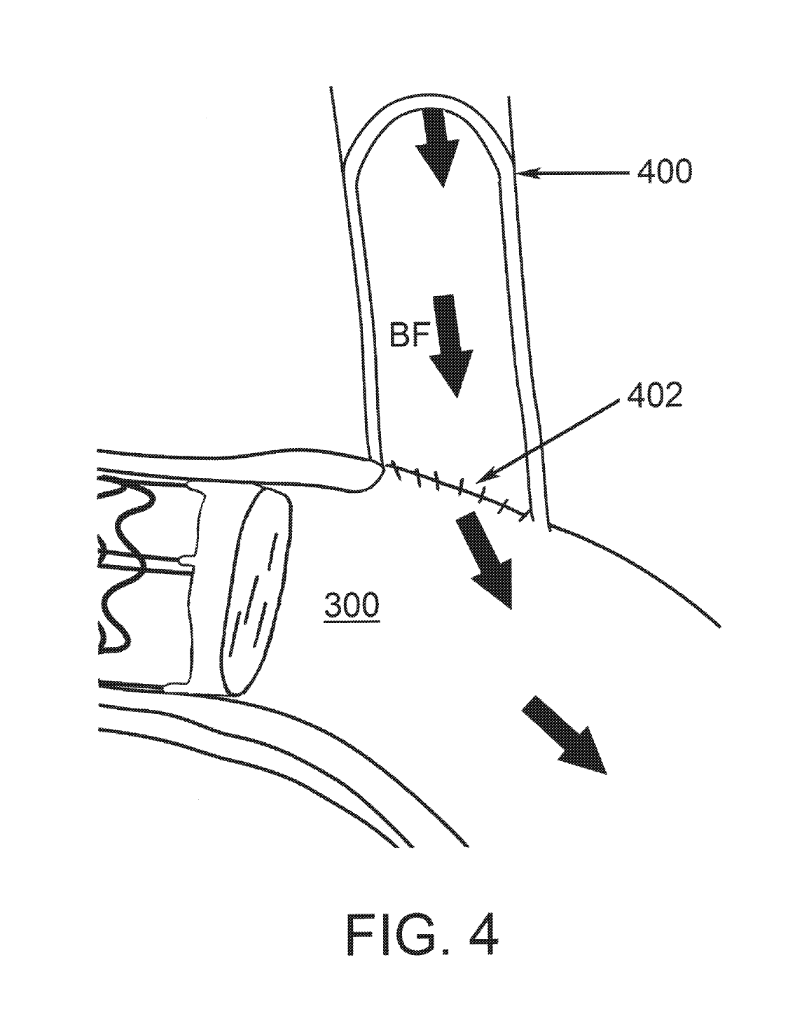

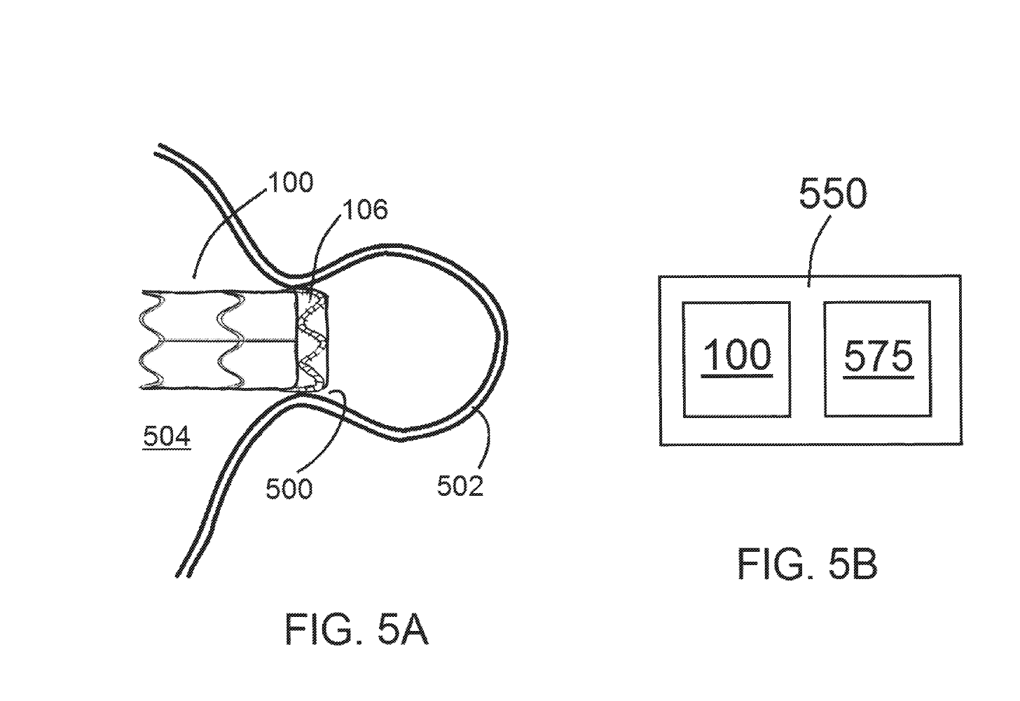

As referenced herein, only one device 100 may be needed to perform the desired procedure, as opposed to using several coils or other occluders, for example. Use of one device 100, with its ease of delivery (such as with a delivery catheter or other delivery device) can not only reduce overall costs as opposed to needing several devices, but it can further reduce the overall time of the procedure to place one device 100 versus several other occluders. Said devices 100 limit flow at specific localized points, allowing a crisp, well defined point of thrombosis to occur while blood can continue to flow through an adjacent blood vessel as shown in FIG. 3, or effectively blocking an atrial appendage such as shown in FIG. 5A. As shown in FIG. 5A, an exemplary device 100 of the present disclosure may be positioned at an opening 500 of an atrial appendage 502, so that blood can continue to flow through the heart 504 but no longer in and out of atrial appendage 502. Orientation of device 100 in such an embodiment can be in any number of ways, but occluding atrial appendage 502 using expandable occluder 106 would be required to restrict blood flow in and out of atrial appendage 502 (such as a left atrial appendage (LAA), for example). This is particularly beneficial for atrial fibrillation patients that are prone to thrombus generation in atrial appendage 502, as identified in U.S. patent application Ser. No. 12/522,674 of Kassab et al., entitled “DEVICES, SYSTEMS, AND METHODS FOR PERCUTANEOUS TRANS-SEPTAL LEFT ATRIAL APPENDAGE OCCLUSION.” As referenced therein, it has been demonstrated by means of echocardiography and autopsy studies that more than 90% of all thrombi in patients with non-rheumatic atrial fibrillation (AF) beginning in the left atrium, appear in the left atrial appendage, and that thrombus formation elevates the threat of stroke by three-fold. As such, occluding atrial appendage 502 using an exemplary device 100 of the present disclosure may reduce the risk of stroke due to thrombus release from atrial appendage 502.

As noted generally above, exemplary devices 100 of the present disclosure may be delivered intravascularly to a location of interest. Said devices 100 may be delivered by a delivery device 575, such as referenced in the component block diagram shown in FIG. 5B, and may comprise a delivery catheter, a delivery wire, or some sort of other delivery device. An exemplary system 550 of the present disclosure, shown in FIG. 5B, comprises an exemplary device 100 and a delivery device 575.

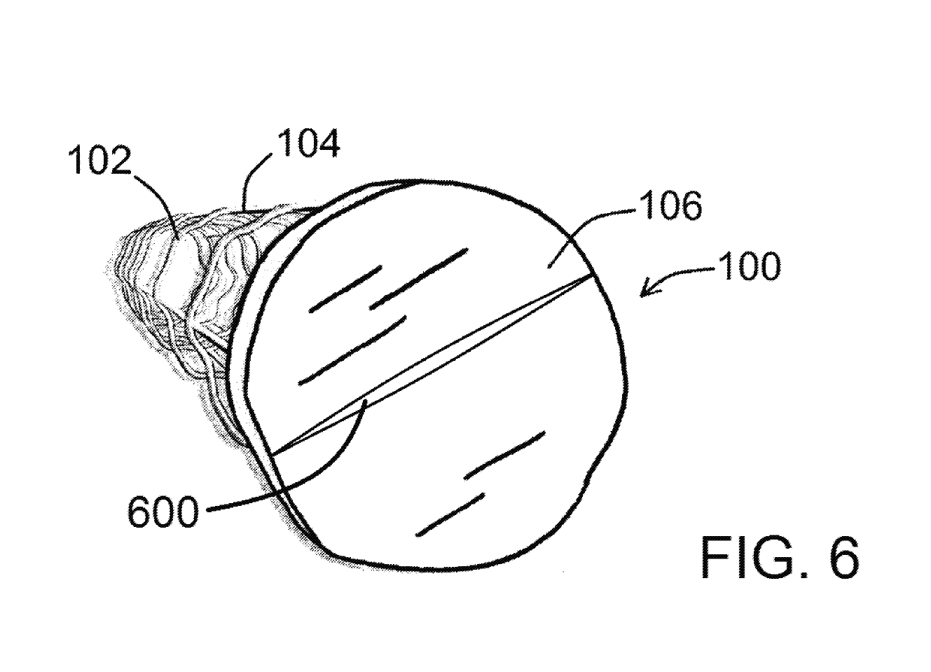

An additional embodiment of an exemplary device 100 of the present disclosure in a generally expanded or deployed configuration is shown in FIG. 6. As shown therein, expandable occluder 106 is configured to span an entire cross-sectional area (CSA) of device 100 at the location of expandable occluder 106. Expandable occluder 106, in such an embodiment, has a self-sealing element 600 defined therein, such as a one-way valve or other element defined within expandable occluder 106 itself, to facilitate the following.

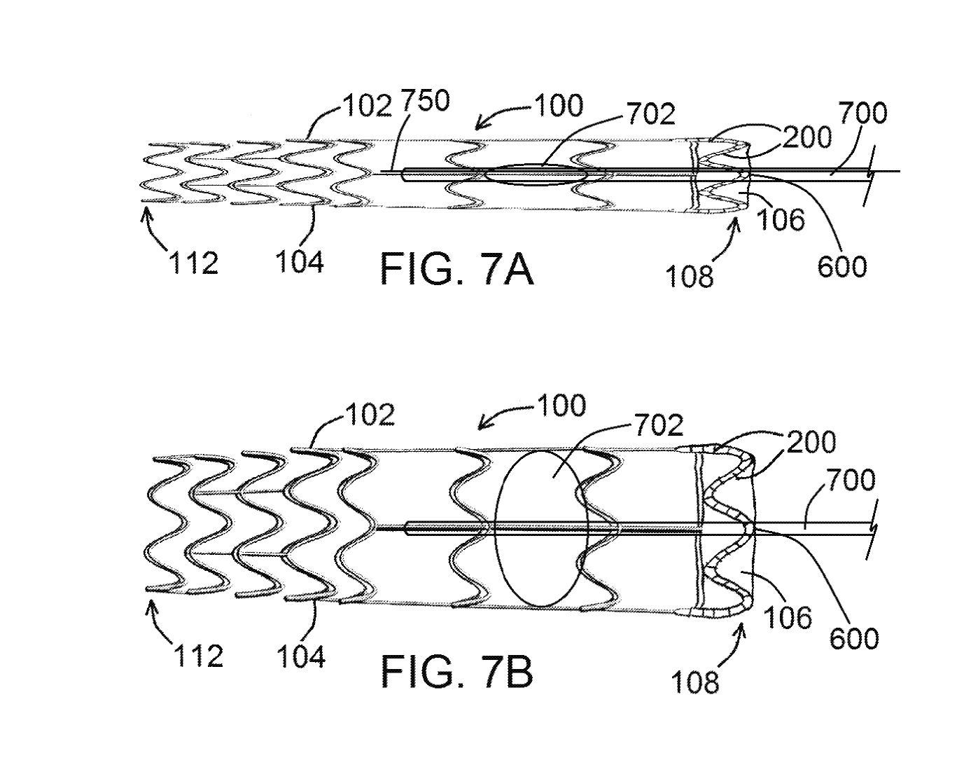

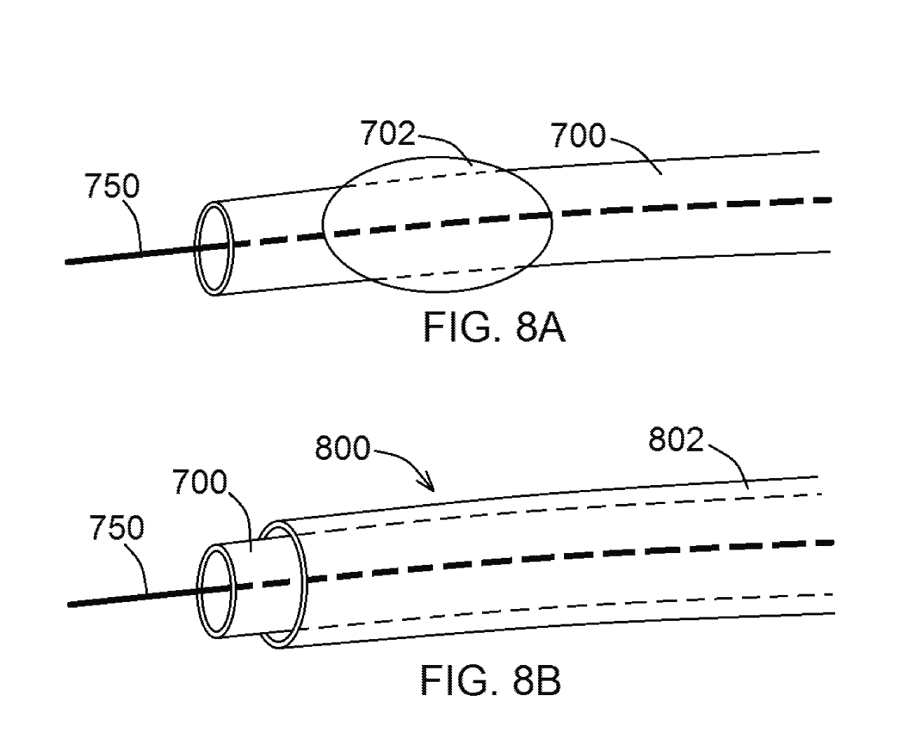

Self-sealing element 600, if defined within expandable occluder 106, would permit a delivery cannula 700, as shown in FIGS. 7A and 7B, to be positioned in part within self-sealing element 600 so that part of delivery cannula 700 is positioned within device 100 during delivery. Procedurally, device 100 could be delivered using a delivery system 800, as referenced in FIG. 8B, comprising a sheath 802 and a delivery cannula 700, whereby device 100 is at least partially positioned within sheath 802 while a distal portion of delivery cannula 700 is positioned within device 100. When device 100 is positioned at a desired location within a vasculature, sheath 802 can be at least partially withdrawn so to reveal device, which can either self-expand, in various embodiments, or expand by way of inflation of a balloon 702 coupled to delivery cannula 700, such that balloon 702 presses against portions of device 100 to cause device 100 to expand within the vasculature. After expansion/implantation of device 100, delivery cannula 700 can be withdrawn from the lumen of device 100 out of self-sealing element 600, whereby self-sealing element 600 can self-seal to occlude as desired. Remaining portions of delivery system 800 can be withdrawn from the vasculature as desired.

Device 100 delivery can also be facilitated using an elongated wire 750, for example, as shown in FIG. 7A. Wire 750 can be inserted into and navigated through a vasculature as desired, and delivery cannula 700 with a sheath 802 thereon, such as shown in FIG. 8B, can be advanced over said wire 750 so to deliver device 100 within sheath 802, for example.

FIG. 7A shows a wire 750 and a delivery cannula 700 at least partially positioned within device 100. Balloon 702, shown in FIG. 7A, is deflated or relatively deflated. Inflation of balloon 702 within device 100, such as shown in FIG. 7B, can cause device 100 to expand so to implant device 100 within the vasculature, as desired.

Device 100 delivery can also be performed using a system 800 such as shown in FIG. 8B, whereby, for example, device is positioned within sheath 802 distal to a distal end of delivery cannula 700, wherein when the distal portion of system 800 having device 100 therein is at a desired location within a vasculature, movement of delivery cannula 700 in a distal direction relative to sheath 802 causes a distal end of delivery cannula 700 to push device 100 out of a distal end of sheath 802 so to deliver device 100. An outer perimeter of delivery cannula 700 can push against an outer perimeter of occluder 106 of device, for example, so not to cause damage to occluder 106 during delivery/implantation.

Regarding device 100 embodiments that are self-expandable, a balloon 702 would not be required as an active element of delivery cannula 700. System 800, comprising a delivery cannula 700 and a sheath 802, for example, could deliver the device 100 by way of moving delivery cannula 700 relative to sheath 802 so to expel device 100 from system 800 as noted above.

In at least one embodiment, the wire 750 guided/contained cannula 700 is removed through the self-sealing element 600 following self or balloon 702 expansion of the device 100. In such a procedure, cannula 700 can be withdrawn through the relative center of device 100, which can be important to controlled guidance and delivery of device 100.

Wire 750, in various embodiments, can be used to center device 100, such that wire 750, when at least partially positioned within delivery cannula 700, keeps system 800 in axial alignment with the vessel and also keeps device 100 in axial alignment during deployment.

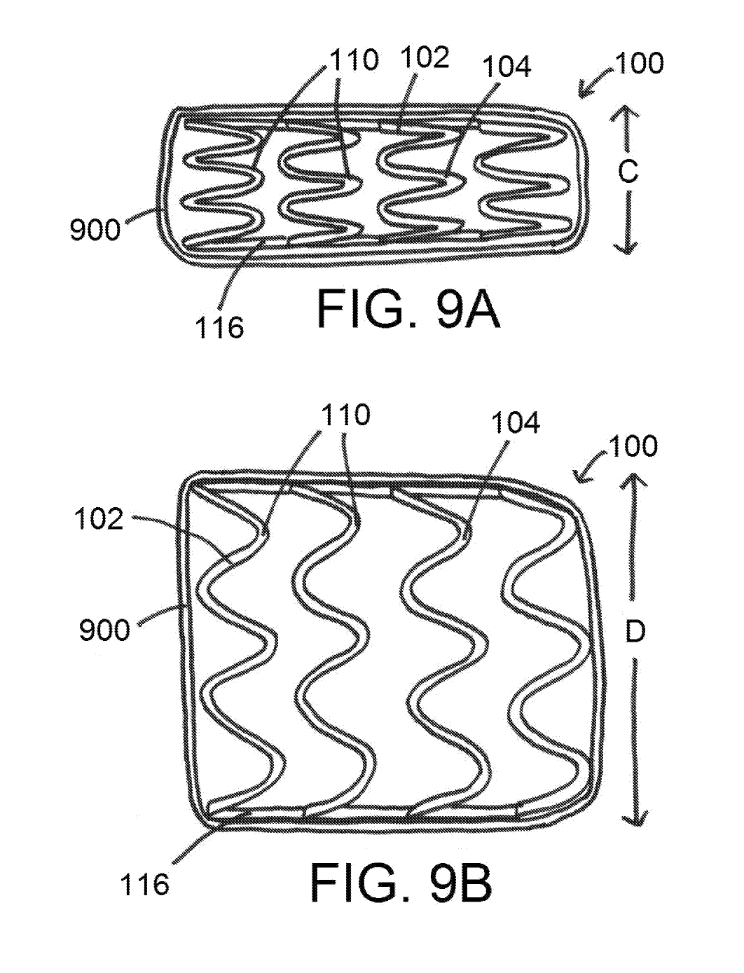

The present disclosure also includes disclosure of additional devices 100, comprising (as noted in further detail below) a stent, or other auto-expandable member, within a balloon. For example, and as shown in FIGS. 9A and 9B, an exemplary device 100 of the present disclosure comprises a frame 102 configured for autoexpansion (also referred to herein as a stent), whereby frame 102 is surrounded by a covering 900, which could be considered as being a balloon. Frame 102 could have any number of struts 104, 110, 116, as referenced herein, so that frame 102 could expand from a compressed/collapsed configuration (as shown in FIG. 9A) to an expanded configuration (as shown in FIG. 9B). Covering 900, made of an impermeable material (such as a rubber or other impermeable biologically-compatible material), would be positioned around frame 102 so that covering 900 covers at least an external perimeter (such as the dimension P shown in FIG. 1) of frame 102 when collapsed or expanded.

Device 100 could be delivered as referenced herein, whereby delivery occurs while device 100 is in a compressed/collapsed configuration, such as shown in FIG. 9A, and is expanded when positioned at a desired location, such as shown in FIG. 9B, whereby a relative first diameter or distance (labeled as “C” in FIG. 9A) enlarges to a second diameter or distance (labeled as “D” in FIG. 9B). Overall expansion size can be controlled by way of size, shape, and/or configuration of frame 102 (the “stent”), and/or the size, shape, compliance, and/or configuration of covering 900 (the “balloon”). For example, a frame 102 would expand more within a covering 900 that is more compliant/stretchable than it would be able to expand in a more rigid covering 900. A frame 102 configured to expand further (such as from “C” to a relatively large “D”) than a frame 102 configured to expand not as much could be chosen, or vice versa, depending on desired configuration. Although covering 900, in such an embodiment, is not “inflated” like a balloon (via gas and/or a fluid), other embodiments could also permit covering 900 “inflation” using a gas and/or a fluid if desired. However, using frame 102 instead of a gas and/or a fluid to expand device 100, one major problem is overcome, namely the tendency for a traditional inflated balloon to deflate or leak over time, such as due to rupture, fluid decompression, etc., as frame 102 supports covering 900 and keeps device 100 expanded as desired.

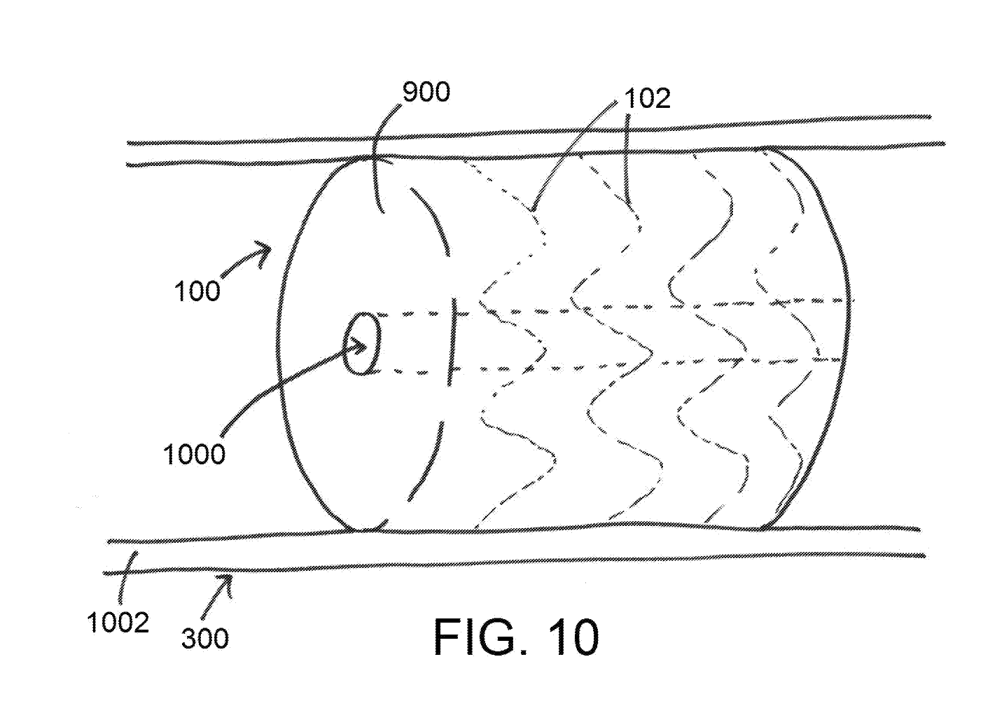

Such an expansion of device 100 could, for example, be used to totally occlude, substantially occlude, or at least partially occlude, a luminal organ 300 of interest, depending on configuration of device 100. For example, a device 100 completely surrounded by a covering 900 could be expanded within a liminal organ 300 to fully occlude the same, such as shown in the device 100 embodiments shown in FIGS. 3 and 5A. Other device 100 embodiments could be configured so to define a passageway 1000 therethrough, such as shown in FIG. 10, so that upon expansion of device 100, passageway 1000 is open so to allow fluid (such as blood) to continue to travel through luminal organ 300 at the location of device 100 therein. FIG. 10 shows an expanded device 100, whereby covering 900 not only covers substantially all of an outer portion of frame 102, but also is used to define the boundary of passageway 1000. Said passageway, such as shown in FIG. 10, may extend from a first end (distal end 108) of device 100 to a second end (proximal end 112) of device 100.

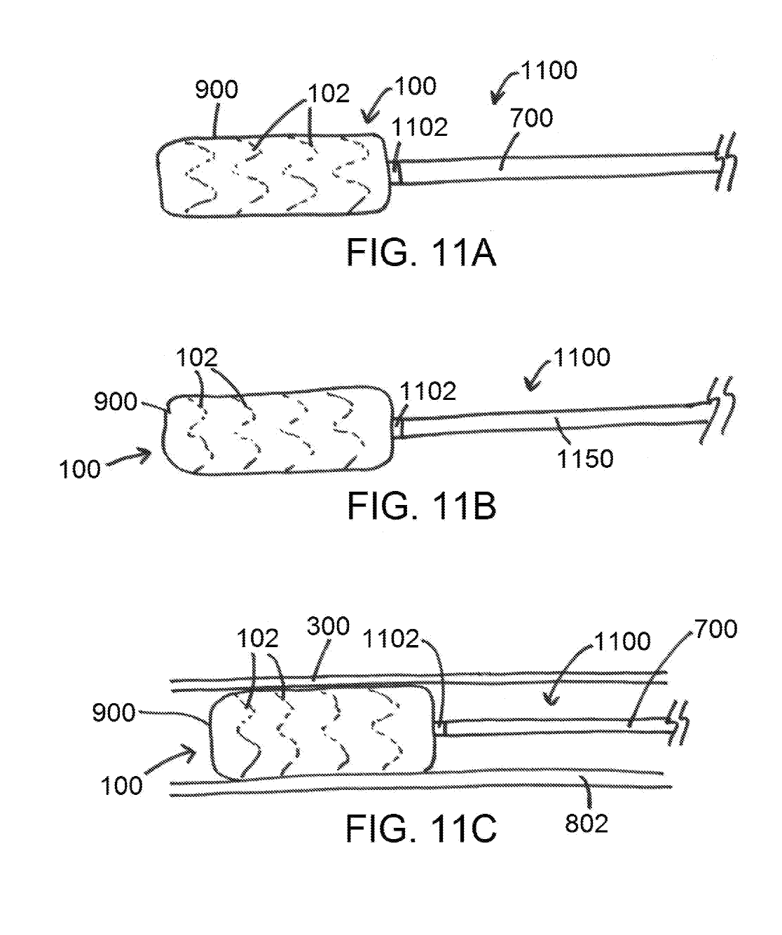

FIG. 11 shows an embodiment of an exemplary overall system 1100 of the present disclosure. An exemplary overall system 1100 (also referred to as an occlusion system 1100) of the present disclosure, such as shown in FIG. 11, comprises an exemplary device 100 and at least part of a delivery system 800 of the present disclosure, such as a delivery cannula 700 shown in the figure. Delivery cannula 700 can be used to place device 100 within a luminal organ 300 of interest, and ultimately be detached therefrom as may be desired/necessary, so that device 100 remains within luminal organ 300 in an expanded configuration. Detachment may occur via use of a detachment member 1102, such as a hook, coil, flange, magnet, etc., configured to engage device 100 and ultimately disengage device 100 as may be desired.

Detachment member 1102 may be formed as part of delivery cannula 700, be attached to delivery cannula 700, or be formed as part of or attached to a delivery shaft 1150, such as shown in FIG. 11B. Delivery shaft 1150, as referenced herein, may have a solid core (no lumen therethrough), while delivery cannula 700, as referenced herein, may be hollow (having a lumen therethrough).

In various embodiments, device 100 could be delivered using a delivery system 800, as referenced in FIG. 11C, comprising a sheath 802 and a delivery cannula 700 (or a delivery shaft 1150, as shown in FIG. 11B), whereby device 100 is at least partially positioned within sheath 802 during delivery while a distal portion of delivery cannula 700 or delivery shaft 1150 is positioned within sheath 802. When device 100 is positioned at a desired location within a vasculature, sheath 802 can be at least partially withdrawn so to reveal device 100, which can self-expand, as referenced herein, within the vasculature. After expansion/implantation of device 100, delivery cannula 700 or delivery shaft 1150 can be detached from device 100. Remaining portions of delivery system 800 can be withdrawn from the vasculature as desired.

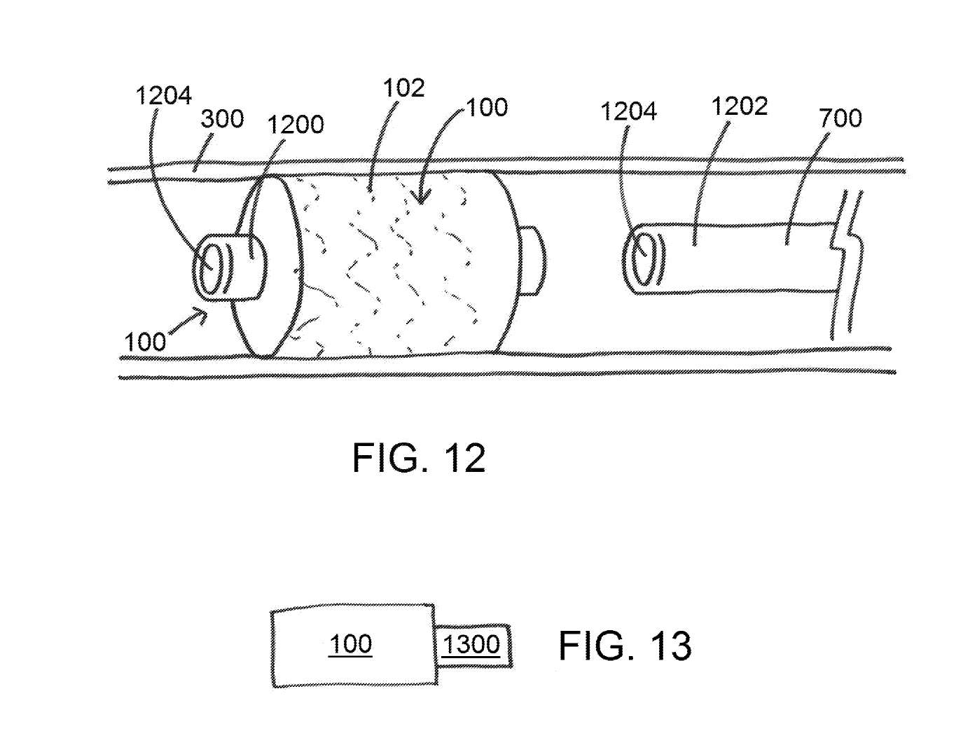

Portions of delivery cannula 700 or delivery shaft 1150, as referenced herein, can remain attached to and/or be positioned within portions of device 100 after detachment of the remainder of the same. For example, and as shown in FIG. 12, a distal portion 1200 of delivery cannula 700 could be positioned within device 100 (such as within passageway 1000), such that when the remainder 1202 of delivery cannula 700 is detached from the distal portion of delivery cannula 700, the distal portion 1200 of delivery cannula 700 remains within device 100, so to provide a passageway for fluid to pass therethrough during and/or after device 100 expansion, so to partially, but not fully, occlude the luminal organ 300 having device 100 positioned therethrough. The extent (amount, etc.) of fluid flow through the part of delivery cannula 700 (the distal portion 1200) within or attached to device 100 can be dictated by way of the diameter of a lumen 1204 of delivery cannula 700, for example.

By way of another example, a distal portion of delivery shaft 1150 could be positioned within device 100 or be attached to device 100, such that when the remainder of delivery shaft 1150 is detached from the distal portion of delivery shaft 1150, the distal portion of delivery shaft 1150 remains within device 100 or attached to device 100. The remaining part of delivery shaft 1150 (the part within or attached to device 100) could then, in various embodiments, occlude a passageway 1000 defined within device 100, so to fully occlude the luminal organ 300 having device 100 positioned therein.

FIG. 13 shows, in block diagram form, an exemplary device 100 of the present disclosure having a retrieval feature 1300 defined therein, coupled thereto, or formed as part of device 100. In various embodiments, retrieval feature(s) 1300 may comprise a hook, a coil, a flange, a magnet, a pocket, etc., and/or be an aperture defined within part of device 100, so that a device used to retrieve device 100 after implantation/expansion, such as a delivery cannula 700, a delivery shaft 1150, a wire 750, or another device (collectively referred to as exemplary “retrieval devices”) can be used to retrieve device by way of engaging retrieval feature 1300. For example, delivery cannula 700, delivery shaft 1150, wire 750, or another device, could also have a retrieval feature 1300 defined therein, coupled thereto, or formed as part of said device, whereby retrieval feature 1300 of device 100 and the device used to retrieve device 100 could engage one another so that device 100 could be withdrawn from the luminal organ 300 or otherwise moved within the body within the same or to a different luminal organ 300 as may be desired.

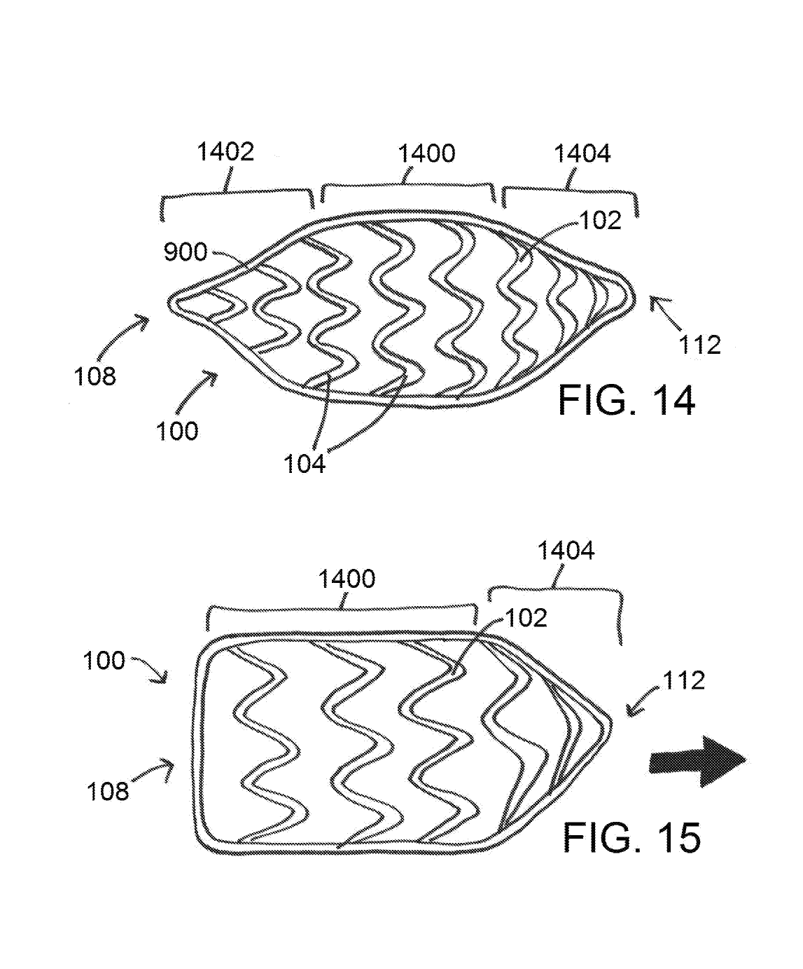

An additional exemplary device 100 of the present disclosure is shown in FIG. 14. As shown therein, device 100 comprises a frame 102 having various struts 104, whereby frame 102 is surrounded by covering 900, as shown in other device 100 embodiments referenced herein. In at least the embodiment shown in FIG. 14, device 100 may have a main portion 1400, which is the portion of device 100 having the largest expanded cross-sectional area, a distal portion 1402, which generally tapers inward from main portion 1400 toward distal end 108, and a proximal portion 1404, which generally tapers inward from main portion 1400 toward proximal end 112. Various devices 100 of the present disclosure may have a distal portion 1402 and/or a proximal portion 1404, and in embodiments where device 100 has only a main portion 1400 and one of a distal portion 1402 or a proximal portion 1404, main portion 1400 would not be “central” to distal portion 1402 and proximal portion 1404 as both of said portions 1402, 1404 would not exist. Main portion 1400, as shown in FIG. 14, would be the portion having portions of frame 102 expand to the fullest extent, while distal portion 1402 and/or proximal portion 1404 would have portions of frame 102 that would expand to a lesser extent, so to permit and/or define the distal and/or proximal tapering.

FIG. 15 shows a device 100 embodiment of the present disclosure having various features/components as referenced herein, but also defining/having a main portion 1400 adjacent to a proximal portion 1404, whereby proximal portion 1404 tapers inward toward proximal end 112. Such a device 100 embodiment would allow for easier removal than an embodiment not having a tapered proximal portion 1404, as tapered proximal portion 1404 would facilitate removal in a proximal direction (direction as shown in the bold arrow in FIG. 15), such as when a retrieval feature 1300 of a delivery cannula, wire 750 or delivery shaft 1150 is used to engage a corresponding detachment member 1102 of device 100 or other portions of device 100 itself.

While various embodiments of devices and systems useful to precondition, arterialize, and/or occlude a mammalian luminal organ and methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device, comprising:
a frame comprising a plurality of struts and configured for expansion; and
a covering surrounding at least an external perimeter of the frame, the covering comprising an impermeable material;
wherein the device is configured for expansion without requiring the introduction of a gas and/or a liquid therein.

2. The device of claim 1, configured to at least partially occlude a mammalian luminal organ at a location of the device when the device is positioned within the mammalian luminal organ and when the frame is expanded from a compressed configuration to an expanded configuration.

3. The device of claim 1, wherein the impermeable material comprises rubber.

4. The device of claim 1, wherein the impermeable material comprises a biologically-compatible material.

5. The device of claim 1, whereby expansion of the frame from a compressed configuration to an expanded configuration causes a diameter or distance of the device to increase.

6. The device of claim 1, whereby no fluid is present within the device.

7. The device of claim 1, configured to completely occlude a mammalian luminal organ at a location of the device when the device is positioned within the mammalian luminal organ and when the frame is expanded from a compressed configuration to an expanded configuration.

8. The device of claim 1, defining a passageway therethrough, the passageway extending from a first end to a second end of the device, the passageway configured to allow blood to flow therethrough when the device is positioned within a mammalian luminal organ and expanded to at least partially occlude the mammalian luminal organ at the device.

9. The device of claim 8, wherein a boundary of the passageway is defined by the covering.

10. The device of claim 1, forming part of an overall system, the overall system further comprising:
a delivery cannula configured to attach to and detach from the device.

11. The device of claim 10, wherein the overall system further comprises:
a detachment member coupled to the delivery cannula, the detachment member configured so that the delivery cannula can attach to and detach from the device.

12. The device of claim 10, wherein the delivery cannula comprises a distal portion and a proximal portion, wherein the passageway is configured to receive at least part of the distal portion therethrough, and wherein the distal portion is configured to detach from the proximal portion after the device is positioned within the mammalian luminal organ.

13. The device of claim 1, further comprising:
a retrieval feature coupled thereto or formed therein, the retrieval feature configured for engagement by a retrieval device, whereby the retrieval device can engage the retrieval feature of the device after the device has been positioned within a mammalian luminal organ so to retrieve the device from the mammalian luminal organ.

14. The device of claim 1, comprising a main portion and a proximal portion, whereby the main portion would define a largest expanded cross-sectional area, and whereby the proximal portion generally tapers inward from the main portion toward a proximal end of the device.

15. The device of claim 14, further comprising a distal portion, the distal portion generally tapering inward from the main portion toward a distal end of the device.

16. A device, comprising:
a frame comprising a plurality of struts and configured for expansion; and
a covering surrounding at least an external perimeter of the frame, the covering comprising an impermeable rubber material;
wherein the device is configured for expansion without requiring the introduction of a gas and/or a liquid therein; and
wherein the device is configured to at least partially occlude a mammalian luminal organ at a location of the device when the device is positioned within the mammalian luminal organ and when the frame is expanded from a compressed configuration to an expanded configuration.

17. The device of claim 16, defining a passageway therethrough, the passageway extending from a first end to a second end of the device, the passageway configured to allow blood to flow therethrough when the device is positioned within a mammalian luminal organ and expanded to at least partially occlude the mammalian luminal organ at the device, and wherein a boundary of the passageway is defined by the covering.

18. A method, comprising:
introducing at least part of a device into a mammalian luminal organ, the device comprising:
a frame comprising a plurality of struts and configured for expansion, and
a covering surrounding at least an external perimeter of the frame, the covering comprising an impermeable material,
wherein the device is configured for expansion without requiring the introduction of a gas and/or a liquid therein; and
expanding the frame to at least partially occlude the mammalian luminal organ.

19. The method of claim 18, wherein the step of introducing is performed by introducing the at least part of the device into the mammalian luminal organ using a cannula coupled to the device.

20. The method of claim 18, further comprising the step of:
retrieving the at least part of the device from the mammalian luminal organ using a retrieval device.
